(19) United States
(12) Patent Application Publication
(10) Pub. No.: US 2019/0015396 A1
(43) Pub. Date: Jan. 17, 2019

(54) VMAT2 INHIBITORS FOR TREATING NEUROLOGICAL DISEASES OR DISORDERS

(71) Applicant: Neurocrine Biosciences, Inc., San Diego, CA (US)

(72) Inventor: Christopher F. O'BRIEN, San Diego, CA (US)

(21) Appl. No.: 15/738,537

(22) PCT Filed: Jun. 23, 2016
(86) PCT No.: PCT/US2016/039098

§ 371 Date: Dec. 20, 2017

Related U.S. Application Data
(60) Provisional application No. 62/183,519, filed on Jun. 23, 2015.
(60) Provisional application No. 62/183,520, filed on Jun. 23, 2015.
(60) Provisional application No. 62/183,525, filed on Jun. 23, 2015.
(60) Provisional application No. 62/183,530, filed on Jun. 23, 2015.
(60) Provisional application No. 62/248,797, filed on Oct. 30, 2015.
(60) Provisional application No. 62/248,803, filed on Oct. 30, 2015.
(60) Provisional application No. 62/251,007, filed on Nov. 4, 2015.
(60) Provisional application No. 62/251,009, filed on Nov. 4, 2015.
(60) Provisional application No. 62/251,012, filed on Nov. 4, 2015.
(60) Provisional application No. 62/251,018, filed on Nov. 4, 2015.
(60) Provisional application No. 62/251,019, filed on Nov. 4, 2015.
(60) Provisional application No. 62/251,023, filed on Nov. 4, 2015.
(60) Provisional application No. 62/262,856, filed on Dec. 3, 2015.
(60) Provisional application No. 62/262,860, filed on Dec. 3, 2015.
(60) Provisional application No. 62/290,839, filed on Feb. 3, 2016.
(60) Provisional application No. 62/290,864, filed on Feb. 3, 2016.

(51) Int. Cl. A61K 31/437; A61P 25/18; A61K 9/20

(52) U.S. Cl. CPC A61K 31/437; A61K 9/20; A61P 25/18

(57) ABSTRACT

Methods are provided herein for treating agitation in a subject who has Alzheimer's disease comprising administering a VMAT2 inhibitor to a subject in need thereof. VMAT2 inhibitors useful in the methods provided herein include tetrabenazine and (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester.

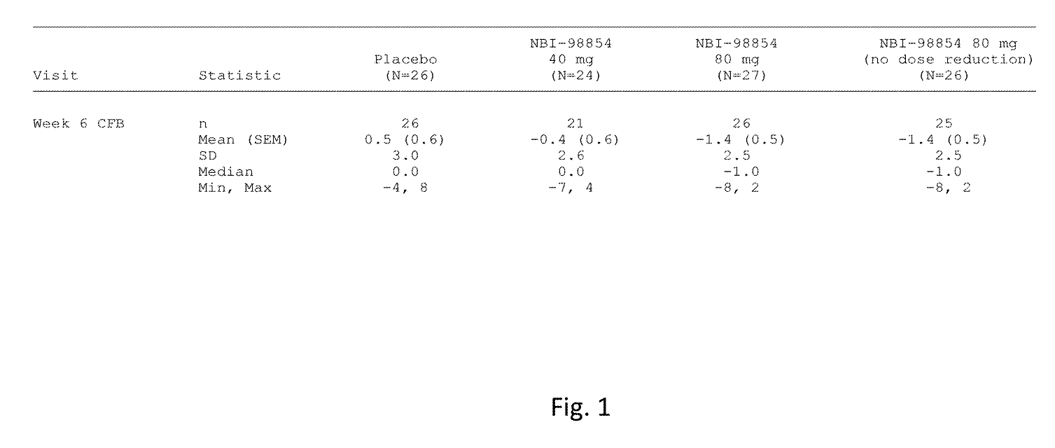

### BACKGROUND

### Technical Field

Provided herein are methods of treating agitation associated with Alzheimer's disease by administering to a subject in need thereof a VMAT2 inhibitor or a pharmaceutical composition comprising the VMAT2 inhibitor.

### Description of the Related Art

Agitation in Alzheimer's disease refers to a cluster of several behavioral symptoms associated with the disease. Agitation develops as the disease progresses and occurs in addition to cognitive loss. The cluster of symptoms includes anxiety, depression, irritability, and motor restlessness (such as pacing, wandering, constant movement). Other symptoms that may occur include sleep disturbances, delusions, hallucinations, compulsive behaviors, aggression, and general emotional distress. Agitation may occur in as many as half of all individuals with Alzheimer's disease. Agitation is associated with patients who have a poor quality of life, deteriorating family relationships and professional caregivers, ultimately leading to admission to a residential care facility.

Patients with Alzheimer's disease and who exhibit agitation have been treated with atypical antipsychotics (e.g., risperidone, olanzapine) and typical antipsychotics (e.g., haloperidol) with only modest success and with risk of serious side effects. Accordingly, a need exists to identify and develop more effective therapeutic agents for treating agitation in patients with Alzheimer's.

### BRIEF SUMMARY

Briefly, this disclosure relates to use of a VMAT2 inhibitor for treating agitation in Alzheimer's disease (also referred to herein as agitation associated with Alzheimer's disease). Provided herein are new methods of treating agitation in a subject who has Alzheimer's disease by administering a VMAT2 inhibitor. The present disclosure provides the following embodiments.

### Embodiment 1

A method for treating agitation in a subject who has Alzheimer's disease comprising administering to the subject a VMAT2 inhibitor.

### Embodiment 2

The method of Embodiment 1, wherein the VMAT2 inhibitor is tetrabenazine (3-isobutyl-9,10-dimethoxy-1,3,4,6,7,11b-hexahydro-2H-pyrido[2,1-a]isoquinolin-2-one) or a pharmaceutically acceptable salt thereof.

### Embodiment 3

The method of Embodiment 1, wherein the VMAT2 inhibitor is (2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof or a pharmaceutically acceptable salt thereof.

### Embodiment 4

The method of Embodiment 1, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or a pharmaceutically acceptable salt thereof.

### Embodiment 5

The method of Embodiment 1, wherein the VMAT2 inhibitor is [(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof or a pharmaceutically acceptable salt thereof.

### Embodiment 6

The method of Embodiment 1, wherein the VMAT2 inhibitor is 3-isobutyl-9,10-d₆-dimethoxy-1,3,4,6,7,11b-hexahydro-2H-pyrido[2,1-a]isoquinolin-2-one (d₆-TBZ) or a pharmaceutically acceptable salt thereof.

### Embodiment 7

A pharmaceutical composition for use in treating agitation in Alzheimer's disease, said composition comprising a pharmaceutically acceptable excipient and a VMAT2 inhibitor.

### Embodiment 8

The pharmaceutical composition of Embodiment 7, wherein the VMAT2 inhibitor is tetrabenazine (3-isobutyl-9,10-dimethoxy-1,3,4,6,7,11b-hexahydro-2H-pyrido[2,1-a]isoquinolin-2-one) or a pharmaceutically acceptable salt thereof.

### Embodiment 9

The pharmaceutical composition of Embodiment 7, wherein the VMAT2 inhibitor is (2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof or a pharmaceutically acceptable salt thereof.

### Embodiment 10

The pharmaceutical composition of Embodiment 7, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or a pharmaceutically acceptable salt thereof.

### Embodiment 11

The pharmaceutical composition of Embodiment 7, wherein the VMAT2 inhibitor is [(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof or a pharmaceutically acceptable salt thereof. Embodiment 12. The pharmaceutical composition of Embodiment 7, wherein the VMAT2 inhibitor is 3-isobutyl-9,10-d₆-dimethoxy-1,3,4,6,7,11b-hexahydro-2H-pyrido[2,1-a]isoquinolin-2-one (d₆-TBZ) or a pharmaceutically acceptable salt thereof.

In an embodiment of the methods described above and herein, the VMAT2 inhibitor is tetrabenazine (3-isobutyl-9,10-dimethoxy-1,3,4,6,7,11b-hexahydro-2H-pyrido[2,1-a]isoquinolin-2-one) or a pharmaceutically acceptable salt thereof. In another specific embodiment, the VMAT2 inhibitor is (2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof or a pharmaceutically acceptable salt thereof. In yet another specific embodiment, the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester. In another specific embodiment the VMAT2 inhibitor is a pharmaceutically acceptable salt of (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, e.g., ditosylate salt. In another embodiment, the VMAT2 inhibitor is deuterated tetrabenazine, particularly 3-isobutyl-9,10-d₆-dimethoxy-1,3,4,6,7,11b-hexahydro-2H-pyrido[2,1-a]isoquinolin-2-one (d₆-TBZ) or a pharmaceutically acceptable salt thereof. In another embodiment, the VMAT2 inhibitor is [(2R,3S,11bR)-9,10-dimethoxy-3-(2-methylpropyl)-1H,2H,3H,4H,6H,7H,11bH-pyrido[2,1-a]isoquinolin-2-yl]methanol or a precursor thereof or a pharmaceutically acceptable salt thereof.

In still other particular embodiments, the methods and uses described herein comprise administering a pharmaceutical composition comprising at least one pharmaceutically acceptable excipient and a VMAT2 inhibitor These and other aspects of the invention will be apparent upon reference to the following detailed description. To this end, various references are set forth herein that describe in more detail certain background information, procedures, compounds and/or compositions, and are each hereby incorporated by reference in their entirety.

Terms not specifically defined herein should be given the meanings that would be given to them by one of skill in the art in light of the disclosure and the context. As used in the specification, however, unless specified to the contrary, the terms have the meaning indicated.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise. Thus, for example, reference to “a non-human animal” may refer to one or more non-human animals, or a plurality of such animals, and reference to “a cell” or “the cell” includes reference to one or more cells and equivalents thereof (e.g., plurality of cells) known to those skilled in the art, and so forth. When steps of a method are described or claimed, and the steps are described as occurring in a particular order, the description of a first step occurring (or being performed) “prior to” (i.e., before) a second step has the same meaning if rewritten to state that the second step occurs (or is performed) “subsequent” to the first step. The term “about” when referring to a number or a numerical range means that the number or numerical range referred to is an approximation within experimental variability (or within statistical experimental error), and thus the number or numerical range may vary between 1% and 15% of the stated number or numerical range. It should also be noted that the term “or” is generally employed in its sense including “and/or” unless the content clearly dictates otherwise. The term, “at least one,” for example, when referring to at least one compound or to at least one composition, has the same meaning and understanding as the term, “one or more.”

### DETAILED DESCRIPTION

As disclosed herein, unexpectedly a VMAT2 inhibitor may be used in methods for treating agitation associated with Alzheimer's disease in subjects in need thereof. Agitation is a cluster of related symptoms, including anxiety, irritability, and motor restlessness that can lead to aggression, shouting, wandering, and pacing (see, e.g., Howard et al., Int. J. Geriatr. Psychiatry 16:714-17 (2001)). Treatment with a VMAT2 inhibitor may reduce the level or degree of any one or more of the symptoms that typify agitation (e.g., anxiety, irritability, and motor restlessness). Administration of a VMAT2 inhibitor may also prevent (i.e., reduce the likelihood of occurrence), reduce frequency of occurrence, or reduce severity of one or more symptoms that are included in the cluster of symptoms of agitation in Alzheimer's disease. Several aspects of agitation associated with Alzheimer's disease are particularly amenable to VMAT2 inhibition based on neuropharmacology of the applicable neural circuitry including the movement disorders (e.g., motor restlessness). VMAT2 inhibition results in modulation of the neurotransmitter systems (e.g., dopamine and serotonin), which appear to be central to motor restlessness and, as such, a reduction in frequency and amplitude of the various movement dysfunctions would be measurable on a variety of clinical assessment scales.

VMAT2 Inhibitors

VMAT2 inhibitors (and physiologically acceptable salts thereof) may reduce the supply of monoamines in the central nervous system by inhibiting the vesicular monoamine transporter isoform 2 (VMAT2). Examples of VMAT2 inhibitors and monoamine depletors that may be used in the methods described herein include, for example, tetrabenazine (3-isobutyl-9,10-dimethoxy-1,3,4,6,7,11b-hexahydro-2H-pyrido[2,1-a]isoquinolin-2-one, TBZ). TBZ is approved for the treatment of chorea associated with Huntington's disease. Use of tetrabenazine for the treatment of TD and a variety of hyperkinetic movement disorders has also been described. Tetrabenazine is readily metabolized upon administration to dihydrotetrabenazine (3-isobutyl-9,10-dimethoxy-1,3,4,6,7,11b-hexahydro-2H-pyrido[2,1-a]isoquinolin-2-ol, DHTBZ), with the R,R,R stereoisomer of DHTBZ believed to be the most active metabolite. In certain embodiments, the methods described herein for treating agitation associated with Alzheimer's disease comprise administering (2R,3R,11bR)-3-isobutyl-9,10-dimethoxy-1,3,4,6,7,11b-hexahydro-2H-pyrido[2,1-a]isoquinolin-2-ol (also called R,R,R-DHTBZ herein) or a precursor thereof. Other VMAT2 inhibitors that may be used in the methods and compositions described herein include TBZ analogs and metabolites, reserpine, lobeline and analogs, and compounds described in U.S. Pat. Nos. 8,039,627; 8,357,697; and 8,524,733. In one embodiment, the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester (see U.S. Pat. No. 8,039,627). In another embodiment, the VMAT2 inhibitor is (2R,3R,11bR)-3-isobutyl-9,10-dimethoxy-1,3,4,6,7,11b-hexahydro-2H-pyrido[2,1-a]isoquinolin-2-ol (R,R,R DHTBZ). In still another embodiment, the VMATs inhibitor is [(2R,3S,11bR)-9,10-Dimethoxy-3-(2-methylpropyl)-1H,2H,3H,4H,6H,7H,11bH-pyrido[2,1-a]isoquinolin-2-yl]methanol (also called Compound 5-1 herein), or a precursor thereof (e.g., a prodrug of Compound 5-1). In yet another embodiment, the VMAT2 inhibitor is tetrabenazine or deuterated tetrabenazine. Deuterated tetrabenazine includes 3-isobutyl-9,10-d₆-dimethoxy-1,3,4,6,7,11b-hexahydro-2H-pyrido[2,1-a]isoquinolin-2-one (d₆-TBZ). As described herein, any one of the VMAT2 inhibitors may be combined with a pharmaceutically acceptable excipient, carrier, and/or diluent to form a pharmaceutical composition.

Characterizing the activity of a VMAT2 inhibitor can be readily determined using in vitro methods and animal models described in the art and herein (see, e.g., Teng, et al., J. Neurochem. 71, 258-65, 1998; Near, (1986), Near, (1986), Mol. Pharmacol. 30: 252-57).

Persons skilled in the art readily appreciate that such assays and techniques are performed using appropriate negative controls (e.g., vehicle only, diluent only, etc.) and appropriate positive controls. Conditions for a particular in vitro assay include temperature, buffers (including salts, cations, media), and other components, which maintain the integrity of the test agent and reagents used in the assay, and which are familiar to a person skilled in the art and/or which can be readily determined. Determining the effectiveness of a VMAT2 inhibitor in an animal model is typically performed using one or more statistical analyses with which a skilled person will be familiar. By way of example, statistical analyses such as two-way analysis of variance (ANOVA), Fisher's exact test, and/or Bonferroni Test, may be used for determining the statistical significance of differences between animal groups.

Compounds described herein include all polymorphs, prodrugs, isomers (including optical, geometric and tautomeric), salts, solvates and isotopes thereof. With regard to stereoisomers, VMAT2 inhibitors may have chiral centers and may occur as racemates, racemic mixtures and as individual enantiomers or diastereomers. All such isomeric forms are included, including mixtures thereof. Unless specifically indicated, throughout the specification and the appended claims, a given chemical formula or name shall encompass tautomers and all stereo, optical and geometrical isomers (e.g., enantiomers, diastereomers, E/Z isomers, etc.) and racemates thereof as well as mixtures in different proportions of the separate enantiomers, mixtures of diastereomers, or mixtures of any of the foregoing forms when such isomers and enantiomers exist, as well as salts thereof, including pharmaceutically acceptable salts thereof and solvates thereof such as for instance hydrates including solvates of the free compounds or solvates of a salt of the compound.

As used herein, pharmaceutically (or physiologically) acceptable salts refer to derivatives of the described compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. For example, such salts include acetates, ascorbates, benzenesulfonates, benzoates, besylates, bicarbonates, bitartrates, bromides/hydrobromides, Ca-edetates/edetates, camsylates, carbonates, chlorides/hydrochlorides, citrates, edisylates, ethane disulfonates, estolates esylates, fumarates, gluceptates, gluconates, glutamates, glycolates, glycollylarsnilates, hexylresorcinates, hydrabamines, hydroxymaleates, hydroxynaphthoates, iodides, isothionates, lactates, lactobionates, malates, maleates, mandelates, methanesulfonates, mesylates, methylbromides, methylnitrates, methylsulfates, mucates, napsylates, nitrates, oxalates, pamoates, pantothenates, phenylacetates, phosphates/diphosphates, polygalacturonates, propionates, salicylates, stearates subacetates, succinates, sulfamides, sulfates, tannates, tartrates, teoclates, toluenesulfonates, triethiodides, ammonium, benzathines, chloroprocaines, cholines, diethanolamines, ethylenediamines, meglumines and procaines. Further pharmaceutically acceptable salts can be formed with cations from metals like aluminium, calcium, lithium, magnesium, potassium, sodium, zinc and the like. (see also, e.g., Pharmaceutical Salts, Birge, S. M. et al., J. Pharm. Sci., (1977), 66, 1-19).

In addition, prodrugs are also included with respect to the compounds described herein. Prodrugs are any covalently bonded carriers that release a compound in vivo when such prodrug is administered to a patient. Prodrugs are generally prepared by modifying functional groups in a way such that the modification is cleaved, either by routine manipulation or in vivo, yielding the parent compound. Prodrugs include, for example, compounds as described herein wherein hydroxy, amine, or acid groups are bonded to any group that, when administered to a subject, cleaves to form the hydroxy, amine or acid groups. Thus, representative examples of prodrugs include (but are not limited to) acetate, formate and benzoate derivatives of alcohol and amine functional groups of a compound. Further, in the case of a carboxylic acid (—COOH), esters may be employed, such as methyl esters, ethyl esters, and the like.

The compounds described herein may exist in a continuum of solid states ranging from fully amorphous to fully crystalline. Furthermore, some of the crystalline forms of the compounds may exist as polymorphs. In addition, some compounds may also form solvates with water or other organic solvents. The term solvate is used herein to describe a molecular complex comprising a compound and one or more pharmaceutically acceptable solvent molecules.

The compounds described herein in certain embodiments are pharmaceutically acceptable isotopically labeled compounds wherein one or more atoms are replaced by atoms having the same atomic number but a different atomic mass. Examples include ²H (deuterium) and ³H (tritium) for hydrogen, ¹¹C, ¹³C and ¹⁴C for carbon, ³⁶Cl for chlorine, ¹⁸F for fluorine, ¹²³I and ¹²⁵I for iodine, ¹³N and ¹⁵N for nitrogen, and ³⁵S for sulfur. Examples also include the substitution of deuterium for ¹H, wherein the deuterium(s) are selectively added to the molecule to alter the metabolism of the drug resulting in some enhanced property such as an increased half-life.

Methods of Treatment and Pharmaceutical Preparations and Compositions

Methods are provided herein for treating agitation associated with Alzheimer's disease in a subject who has Alzheimer's disease by administering to the subject in need thereof a VMAT2 inhibitor. The VMAT2 inhibitor may prevent (i.e., reduce likelihood of occurrence of), slow progression of, delay, or treat agitation. Common symptoms of agitation include motor restlessness, physically aggressive behavior, pacing, excessive fidgeting, repetitive behaviors, and abnormal vocalization. In certain embodiments, one or more symptoms of agitation is treated by the methods comprising administering a VMAT2 inhibitor. In a specific embodiment, a method of treating any one or more of anxiety, irritability, pacing, excessive fidgeting, repetitive behaviors, abnormal vocalization, and motor restlessness associated with Alzheimer's disease by administering a VMAT2 inhibitor is provided.

As understood by a person skilled in the medical art, the terms, “treat” and “treatment,” refer to medical management of a disease, disorder, or condition of a subject (i.e., patient) (see, e.g., Stedman's Medical Dictionary). The terms “treatment” and “treating” embraces both preventative, i.e. prophylactic, or therapeutic, i.e. curative and/or palliative, treatment. Thus the terms “treatment” and “treating” comprise therapeutic treatment of patients having already developed the condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods described herein may be used, for instance, as therapeutic treatment over a period of time as well as for chronic therapy. In addition the terms “treatment” and “treating” comprise prophylactic treatment, i.e., a treatment of patients at risk to develop a condition mentioned hereinbefore, thus reducing the risk.

The subject in need of the compositions and methods described herein includes a subject who has been diagnosed by a person skilled in the medical art. Behavioral and psychiatric symptoms of agitation can be diagnosed by a person skilled in the clinical art. Diagnostic tools routinely used by clinicians to diagnose and monitor effectiveness of treatment in a subject with agitation include Neuropsychiatric Inventory (NPI) (see, e.g., Cummings et al., Neurology 44:2308-14 (1994)); and the Cohen-Mansfield Agitation Inventory (CMAI) (see, e.g., Cohen-Mansfield et al., J. Gerontol. 44:M77-M84 (1989); Ballard et al., BMJ 330:874-77 (2005)). A clinician can also eliminate other medical reasons for agitation, such as infection, prescription medications, or uncorrected visual or hearing loss.

A subject (or patient) to be treated may be a mammal, including a human or non-human primate. The mammal may be a domesticated animal such as a cat or a dog.

Therapeutic and/or prophylactic benefit includes, for example, an improved clinical outcome, both therapeutic treatment and prophylactic or preventative measures, wherein the object is to prevent or slow or retard (lessen) an undesired physiological change or disorder, or to prevent or slow or retard (lessen) the expansion or severity of such disorder. As discussed herein, beneficial or desired clinical results from treating a subject include, but are not limited to, abatement, lessening, or alleviation of symptoms that result from or are associated the disease, condition, or disorder to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. “Treatment” can also mean prolonging survival when compared to expected survival if a subject were not receiving treatment. Subjects in need of treatment include those who already have the condition or disorder as well as subjects prone to have or at risk of developing the disease, condition, or disorder (e.g., agitation associated with Alzheimer's disease), and those in which the disease, condition, or disorder is to be prevented (i.e., decreasing the likelihood of occurrence of the disease, disorder, or condition).

A “therapeutically effective amount” generally refers to an amount of a treatment, such as a VMAT2 inhibitor, that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein. Optimal doses may generally be determined using experimental models and/or clinical trials. The optimal dose may depend upon the body mass, weight, or blood volume of the subject. In general, the dose range of a compound that is a VMAT2 inhibitor applicable per day is usually from 5.0 to 150 mg per day, and in certain embodiments from 10 to 100 mg per day. The dose of the VMAT2 inhibitor included in a composition is sufficient to treat agitation associated with Alzheimer's disease (i.e., the dose is a therapeutically effective dose for treating, preventing (i.e., reducing likelihood of occurrence of), slowing progression of, delaying the onset of agitation associated with Alzheimer's disease or one or more symptoms of agitation).

The VMAT2 inhibitor is administered at a time and frequency appropriate for treating agitation associated with Alzheimer's disease. The VMAT2 inhibitor may be administered 1, 2, or 3 times a day. The dose of a VMAT2 inhibitor may be dose-titrated in a subject.

The minimum dose that is sufficient to provide effective therapy and minimize toxicity is usually preferred. Subjects may generally be monitored for therapeutic effectiveness by clinical evaluation and by using assays suitable for the condition being treated or prevented, which assays will be familiar to those having ordinary skill in the art and are described herein. The level of a compound that is administered to a subject may be monitored by determining the level of the compound in a biological fluid, for example, in the blood, blood fraction (e.g., serum), and/or in the urine, and/or other biological sample from the subject. Any method practiced in the art to detect the compound may be used to measure the level of compound during the course of a therapeutic regimen.

The dose of a composition comprising a VMAT2 inhibitor described herein for treating agitation associated with Alzheimer's disease may depend upon the subject's condition, that is, stage of the disease, severity of symptoms caused by the disease, general health status, as well as age, gender, and weight, and other factors apparent to a person skilled in the medical art. Similarly, the dose of the VMAT2 inhibitor compound may be determined according to parameters understood by a person skilled in the art and as described herein.

VMAT2 inhibitor tetrabenazine, which contains two chiral centers and is a racemic mix of two stereoisomers, is rapidly and extensively metabolized in vivo to its reduced form, 3-isobutyl-9,10-dimethoxy-1,3,4,6,7,11b-hexahydro-2H-pyrido[2,1-a]isoquinolin-2-ol, also known as dihydrotetrabenazine (DHTBZ). DHTBZ is thought to exist as four individual isomers: (±)α-DHTBZ and (±) beta-DHTBZ. The (2R, 3R, 11bR)-3-isobutyl-9,10-dimethoxy-1,3,4,6,7,11b-hexahydro-2H-pyrido[2,1-a]isoquinolin-2-ol (R,R,R, DHTBZ) isomer, also known as (±)α-3-isobutyl-9,10-dimethoxy-1,3,4,6,7,11b-hexahydro-2H-pyrido[2,1-a]isoquinolin-2-ol, is believed to be the absolute configuration of the most active metabolite (see, e.g., Kilbourn Chirality 1997 9:59-62).

In one aspect, a method for treating agitation associated with Alzheimer's disease is provided herein that comprises administering to a subject in need thereof a pharmaceutical composition comprising a VMAT2 inhibitor described herein in an amount sufficient to achieve a maximal blood plasma concentration (Cₘₐₓ) of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of between about 15 ng to about 60 ng per mL plasma and a minimal blood plasma concentration (Cₘᵢₙ) of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of at least 15 ng per mL plasma over an 8 hour period.

Reference to plasma concentration of (2R,3R,11bR)-3-isobutyl-9,10-dimethoxy-1,3,4,6,7,11b-hexahydro-2H-pyrido[2,1-a]isoquinolin-2-ol (R,R,R DHTBZ) in the methods described herein includes both deuterated (2R,3R,11bR)-3-isobutyl-9,10-dimethoxy-1,3,4,6,7,11b-hexahydro-2H-pyrido[2,1-a]isoquinolin-2-ol (R,R,R DHTBZ) and non-deuterated (2R,3R,11bR)-3-isobutyl-9,10-dimethoxy-1,3,4,6,7,11b-hexahydro-2H-pyrido[2,1-a]isoquinolin-2-ol (R,R,R DHTBZ). It is apparent to a person of skill in the art that if a deuterated VMAT2 inhibitor as described herein is administered to a subject (e.g., deuterated tetrabenzine, deuterated (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, or deuterated (2R,3R,11bR)-3-isobutyl-9,10-dimethoxy-1,3,4,6,7,11b-hexahydro-2H-pyrido[2,1-a]isoquinolin-2-ol, then deuterated (2R,3R,11bR)-3-isobutyl-9,10-dimethoxy-1,3,4,6,7,11b-hexahydro-2H-pyrido[2,1-a]isoquinolin-2-ol will appear in the subject's blood plasma and is to be measured. If a non-deuterated VMAT2 inhibitor as described herein is administered to a subject (e.g., tetrabenzine, (S)-2-amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester, or (2R,3R,11bR)-3-isobutyl-9,10-dimethoxy-1,3,4,6,7,11b-hexahydro-2H-pyrido[2,1-a]isoquinolin-2-ol, then non-deuterated (2R,3R,11bR)-3-isobutyl-9,10-dimethoxy-1,3,4,6,7,11b-hexahydro-2H-pyrido[2,1-a]isoquinolin-2-ol will appear in the subject's blood plasma and is to be measured. If a combination of deuterated and non-deuterated VMAT2 inhibitors as described herein is administered to a subject, then both deuterated and non-deuterated (2R,3R,11bR)-3-isobutyl-9,10-dimethoxy-1,3,4,6,7,11b-hexahydro-2H-pyrido[2,1-a]isoquinolin-2-ol will appear in the subject's blood plasma and both are to be measured.

In certain embodiments, the Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) is about 15 ng/mL, about 20 ng/mL, about 25 ng/mL, about 30 ng/mL, about 35 ng/mL, about 40 ng/mL, about 45 ng/mL, about 50 ng/mL, about 55 ng/mL or about 60 ng/mL plasma. In certain embodiments, the Cₘᵢₙ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) is at least 15 ng/mL, at least 20 ng/mL, at least 25 ng/mL, at least 30 ng/mL, or at least 35 ng/mL plasma, over a period of 8 hrs, 12 hrs, 16 hrs, 20 hrs, 24 hrs, 28 hrs, or 32 hrs. In certain embodiments, the Cₘᵢₙ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) is between about 15 ng/mL to about 35 ng/mL.

In an embodiment, the pharmaceutical composition is administered in an amount sufficient to provide a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘᵢₙ of approximately at least 33% of the Cₘₐₓ over a 24 hour period. In another embodiment, the pharmaceutical composition is administered in an amount sufficient to provide a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘᵢₙ of approximately at least 50% of the Cₘₐₓ over a 24 hour period. In certain particular embodiments, the pharmaceutical composition is administered in an amount sufficient to provide a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘₐₓ of approximately between about at least 33%-50% of the Cₘₐₓ over a 24 hour period.

In other certain embodiments, the pharmaceutical composition is administered in an amount sufficient to provide a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘᵢₙ of approximately at least 33% of the Cₘₐₓ over a 12 hour period. In yet another certain embodiment, the pharmaceutical composition is administered in an amount sufficient to provide a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘᵢₙ of approximately at least 50% of the Cₘₐₓ over a 12 hour period. In certain particular embodiments, the pharmaceutical composition is administered in an amount sufficient to provide a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘᵢₙ of approximately between about at least 33%-50% of the Cₘₐₓ over a 12 hour period.

In another embodiment, the pharmaceutical composition is administered to a subject in need thereof in an amount that provides a Cₘₐₓ of (2R,3R,11bR)-3-isobutyl-9,10-dimethoxy-1,3,4,6,7,11b-hexahydro-2H-pyrido[2,1-a]isoquinolin-2-ol (R,R,R DHTBZ) of about 15 ng/mL to about 60 ng/mL plasma and a Cₘᵢₙ of between about 5 ng/mL to about 30 ng/mL plasma over a 24 hour period. In yet another embodiment, the pharmaceutical composition is administered to a subject in need thereof in an amount that provides a Cₘₐₓ of (2R,3R,11bR)-3-isobutyl-9,10-dimethoxy-1,3,4,6,7,11b-hexahydro-2H-pyrido[2,1-(R,R,R DHTBZ) of about 15 ng/mL to about 60 ng/mL plasma and a Cₘᵢₙ of between about 7.5 ng/mL to about 30 ng/mL plasma over a 24 hour period.

In another aspect, a method for treating agitation associated with Alzheimer's disease is provided herein that comprises administering to a subject in need thereof a pharmaceutical composition comprising a VMAT2 inhibitor described herein in an amount sufficient to provide: (i) a therapeutic concentration range of about 15 ng to about 60 ng (2R,3R,11bR)-3-isobutyl-9,10-dimethoxy-1,3,4,6,7,11b-hexahydro-2H-pyrido[2,1-a]isoquinolin-2-ol (R,R,R DHTBZ) per mL plasma; and (ii) a threshold concentration of at least 15 ng of (2R,3R,11bR)-3-isobutyl-9,10-dimethoxy-1,3,4,6,7,11b-hexahydro-2H-pyrido[2,1-a]isoquinolin-2-ol (R,R,R DHTBZ) per mL plasma over a period of about 8 hours to about 24 hours.

In certain embodiments, the therapeutic concentration range is about 15 ng to about 35 ng, to about 40 ng, to about 45 ng, to about 50 ng, or to about 55 ng (2R,3R,11bR)-3-isobutyl-9,10-dimethoxy-1,3,4,6,7,11b-hexahydro-2H-pyrido[2,1-a]isoquinolin-2-ol (R,R,R DHTBZ) per mL plasma.

In certain embodiments, the threshold concentration of (2R,3R,11bR)-3-isobutyl-9,10-dimethoxy-1,3,4,6,7,11b-hexahydro-2H-pyrido[2,1-a]isoquinolin-2-ol (R,R,R DHTBZ) is about 15 ng/mL, about 20 ng/mL, about 25 ng/mL, about 30 ng/mL, about 35 ng/mL, about 40 ng/mL, about 45 ng/mL, about 50 ng/mL, about 55 ng/mL or about 60 ng/mL plasma, over a period of about 8 hrs, about 12 hrs, about 16 hrs, about 20 hrs, about 24 hrs, about 28 hrs, or about 32 hrs. In a particular embodiment, the threshold concentration of (2R,3R,11bR)-3-isobutyl-9,10-dimethoxy-1,3,4,6,7,11b-hexahydro-2H-pyrido[2,1-a]isoquinolin-2-ol (R,R,R DHTBZ) is between about 15 ng/mL to about 35 ng/mL over a period of about 8 hours to about 24 hours.

Plasma concentrations of (2R,3R,11bR)-3-isobutyl-9,10-dimethoxy-1,3,4,6,7,11b-hexahydro-2H-pyrido[2,1-a]isoquinolin-2-ol (R,R,R DHTBZ), and compounds as disclosed herein may be measured by methods as described in Derangula et al., Biomedical Chromatography 2013 27(6): 792-801, Mehvar et al., Drug Metabolism and Distribution 1987 15(2): 250-55 and generally by tandem mass spectroscopy.

The pharmaceutical compositions described herein that comprise at least one of the VMAT2 inhibitor compounds described herein may be administered to a subject in need by any one of several routes that effectively deliver an effective amount of the compound. Such administrative routes include, for example, oral, parenteral, enteral, rectal, intranasal, buccal, sublingual, intramuscular, and transdermal. Compositions administered by these routes of administration and others are described in greater detail herein.

Also provided herein are methods for treating agitation, or the neuropsychiatric symptoms of agitation, that are currently treated with antipsychotics. Methods are provided herein for treating agitation by administering to a subject in need thereof a first generation (i.e., typical) or a second generation (i.e., atypical) antipsychotic drug (e.g., a compound) in combination with a VMAT2 inhibitor. In certain embodiments, when the subject has developed a movement disorder that forms the agitation cluster or has at least one symptom of a movement disorder, the methods comprising administering a VMAT2 inhibitor in combination with the antipsychotic are useful for treating the movement disorder.

The dose of the antipsychotic used typically to treat agitation in Alzheimer's disease may be lower than (i.e., reduced, decreased, less than) the heretofore-described dosing range of the drug alone for effectively treating agitation. In certain embodiments, the dose of the antipsychotic drug that is administered when combined with a VMAT2 inhibitor would not effectively treat the neuropsychiatric disorder if administered alone (i.e., if administered in the absence of the VMAT2 inhibitor). In other words, the combination of the VMAT2 inhibitor and the antipsychotic drug act synergistically in the treatment of agitation. When used in combination with a VMAT2 inhibitor, an antipsychotic drug may be used at a dose that if administered alone would have little or no efficacy in treating the neuropsychiatric disorder, that is, the dose of the antipsychotic drug is subtherapeutic. That is, by combining a VMAT2 inhibitor with a subtherapeutic dose of the antipsychotic drug, the efficacy of the antipsychotic drug is enhanced. By way of example, treatment of the neuropsychiatric disorder or symptoms thereof may provide greater relief of agitation and associated anxiety.

Decreasing the dose of an antipsychotic drug has the beneficial effect of reducing the intensity of or preventing (i.e., decreasing the likelihood or risk of occurrence) one or more side effects of the antipsychotic drug. In one embodiment, such as when a typical antipsychotic drug is used for treating agitation, the likelihood of occurrence of movement disorders may be reduced; the severity or intensity of the movement disorder may be decreased or lessened; or the frequency of occurrence of the movement disorder (or symptom thereof) may be reduced (i.e., decreased, lessened). In another embodiment, such as when an atypical drug is used in combination with a VMAT2 inhibitor for treating a neuropsychiatric disorder or symptoms thereof, the likelihood of occurrence or severity of a metabolic disturbance such as weight gain, glucose intolerance, and risk of atherosclerotic cardiovascular disease may be reduced. In other embodiments, side effects that may be reduced by administering to a subject in need thereof an anti-psychotic (either an atypical or typical antipsychotic) combined with a VMAT inhibitor include one or more of sedation, dry mouth, sexual dysfunction, and cardiac arrhythmias.

A typical antipsychotic drug (i.e., first generation antipsychotic drug). includes any one of fluphenazine, haloperidol, loxapine, molindone, perphenazine, pimozide, sulpiride, thioridazine, or trifluoperazine. An atypical antipsychotic drug (i.e., second generation antipsychotic drug) may be any one of aripiprazole, asenapine, clozapine, iloperidone, olanzapine, paliperidone, quetiapine, risperidone, or ziprasidone. The typical antipsychotic haloperidol and the atypical antipsychotics, olanzapine and risperidone have been used for treating patients with Alzheimer's disease with modest benefits observed, however, with increased cognitive decline, cerebrovascular events, parkinsonism, and death (see, e.g., Ballard et al., Nat. Rev. Neurosci. 7:492-500 (2006); Schneider et al., Am. J. Geriatr. Psychiatry 14:191-210 (2006)). Therefore, reduction of the dose of the antipsychotic by administering concurrently a VMAT2 inhibitor could reduce the potential side effects of the antipsychotic as well as treat agitation.

When a VMAT2 inhibitor is administered for treating agitation associated with Alzheimer's disease in combination with an antipsychotic, each of the antipsychotic and the VMAT2 inhibitor are administered at a time and frequency appropriate for treating agitation. The VMAT2 inhibitor may be administered 1, 2, or 3 times a day. The antipsychotic drug may be administered 1, 2, or 3 times a day independently or together with the VMAT2 inhibitor. In other embodiments, the antipsychotic is administered every week, every two weeks (approximately 2 times per month), every three weeks, every four weeks (approximately once per month), every 6 weeks, or every 8 weeks. In particular embodiments, the dose of the antipsychotic drug used in combination with a VMAT2 inhibitor may be at least about 10% less, at least about 20% less, at least about 25% less, at least about 30% less, at least about 35% less, at least about 40% less, at least about 45% less, at least about 50% less, at least about 55% less, at least about 60% less, at least about 65% less, at least about 70% less, at least about 75% less, at least about 80% less, at least about 85% less, or at least about 90% less than when used alone. In other certain embodiments, the dose of the antipsychotic drug when used in combination with a VMAT2 inhibitor may be between 10-90% less, 10-20% less, 10-25% less, 20-30% less, 25%-30% less, 25%-40% less, 25%-50% less, 25%-60% less, 25%-75% less, 25%-80% less, 30-40% less, 30-60% less, 40-50% less, 40-60% less, 50-60% less, 50-75% less, 60-70% less, 60-75% less, 70%-80% less, or 80-90% less than when the antipsychotic drug is used alone.

A VMAT2 inhibitor described herein for treating agitation associated with Alzheimer's disease may be administered in combination with other drugs for treating Alzheimer's disease. By way of example, a VMAT2 inhibitor may be administered in combination with a cholinesterase inhibitor (e.g., RAZADYNE® (galantamine), EXELON® (rivastigmine), and ARICEPT® (donepezil)), an N-methyl D-aspartate (NMDA) antagonist (e.g., NAMENDA® (memantine)), vitamin E, or a combination thereof.

A pharmaceutical composition comprising a VMAT2 inhibitor may further comprise at least one physiologically (or pharmaceutically) acceptable or suitable excipient. Any physiologically or pharmaceutically suitable excipient or carrier (i.e., a non-toxic material that does not interfere with the activity of the active ingredient(s)) known to those of ordinary skill in the art for use in pharmaceutical compositions may be employed in the compositions described herein. Exemplary excipients include diluents and carriers that maintain stability and integrity of the compound.

Pharmaceutically acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, Pa. (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used.

The pharmaceutical compositions may be in the form of a solution. The solution may comprise saline or sterile water, and may optionally include antioxidants, buffers, bacteriostats, and other common additives. Alternatively, they may be in the form of a solid, such as powder, tablets, pills, or the like. A composition comprising any one of the compounds described herein may be formulated for depot injection, sustained or slow release (also called timed release). Such compositions may generally be prepared using well known technology and administered by, for example, oral, rectal or subcutaneous implantation, intramuscular, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of VMAT2 inhibitor compound release. The amount of compound contained within a sustained release formulation depends upon the site of implantation, the rate and expected duration of release, and the nature of the condition to be treated or prevented.

For oral formulations, a VMAT2 inhibitor compound described herein can be used alone or in combination with appropriate additives to make tablets, powders, granules or capsules, for example, dispersing and surface active agents; with conventional additives, such as lactose, mannitol, corn starch or potato starch; with binders; with disintegrators; with lubricants; and if desired, with diluents, buffering agents, moistening agents, preservatives, coloring agents, and flavoring agents. Compounds may be formulated with a buffering agent to provide for protection of the compound from low pH of the gastric environment and/or an enteric coating. A compound included in the compositions may be formulated for oral delivery with a flavoring agent, e.g., in a liquid, solid or semi-solid formulation and/or with an enteric coating. Oral formulations may be provided as gelatin capsules, which may contain the active compound along with powdered carriers, such as lactose, starch, cellulose derivatives, magnesium stearate, stearic acid, and the like. Similar carriers and diluents may be used to make compressed tablets.

Also provided are kits that comprise one or more unit doses of the VMAT2 inhibitor. A non-limiting example of such a kit includes a blister pack.

The following examples are provided for purposes of illustration, not limitation.

### EXAMPLES

### Example 1

### [(2R,3 S,11BR)-9,10-Dimethoxy-3-(2-Methylpropyl)-1H,2H,3H,4H,6H,7H,11 BH-Pyrido[2,1-A]Isoquinolin-2-Yl]Methanol

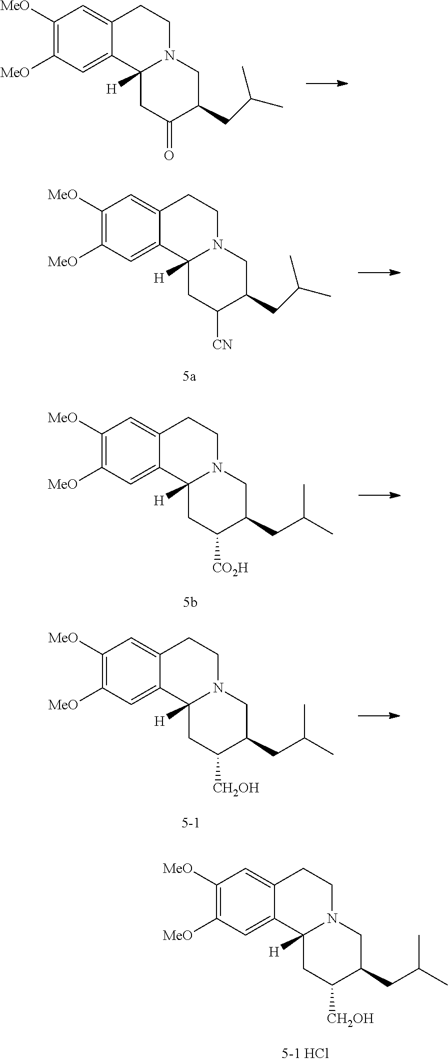

### Step 5A: (3S,11bR)-9,10-Dimethoxy-3-(2-methylpropyl)-1H,2H,3H,4H,6H,7H,11bH-pyrido[2,1-a]isoquinoline-2-carbonitrile

To a 3 L 3-neck round bottomed flask DMSO (1.1 L) and TOSMIC (104 g, 532.5 mmol, 1.3 eq) were charged. To this mixture KO-t-Bu (119.5 g, 1.065 mol) was charged at once at ambient temp (22° C.). An exotherm was observed and the temperature of the mixture increased to 39° C. Then a suspension of tetrabenazine (130 g, 410 mmol) in DMSO (500 mL) was added to the reaction mixture slowly over 25 min (a slight exotherm observed). EtOH (10.5 mL) was added to this mixture, and the mixture was stirred at ambient temp for 3 h. LC-MS analysis of the mixture revealed presence of ˜4:1 ratio of 5a and starting material. The mixture was poured into cold water (9 L). The mixture was then extracted with EtAOc (4 L). The aqueous layer was extracted with EtOAc (2 L). The combined organics were washed with brine (2 L), dried over Na₂SO₄ and concentrated. The residue was dissolved in acetone (200 ml) and loaded onto a silica column (2 Kg silica gel, packed with hexanes). The column was eluted first with hexanes (2.5 L), followed by 5-20% of acetone in hexanes. The fractions containing 5a and other impurities were combined and concentrated to give an orange oil (72 g), which was dissolved in acetone (100 ml) and loaded onto a silica column (1 Kg silica gel, packed with hexanes). The column was eluted first with hexanes (1 L), followed by 5% of acetone in hexanes (2 L), 10% of acetone in hexanes (2 L), 15% of acetone in hexanes (2 L), and 20% of acetone in hexanes (2 L). The fractions containing >90% purity were combined and concentrated to give (3 S,11bR)-9,10-dimethoxy-3-(2-methylpropyl)-1H,2H,3H,4H,6H,7H,11bH-pyrido[2,1-a]isoquinoline-2-carbonitrile 5a as an orange solid (61 g, m/z 329.2 [MH⁺]). The fractions containing a mixture of 5a and starting material were collected and concentrated to give 48 g of material, which was dissolved in DMSO (50 ml) and was added to a mixture of TOSMIC (25 g) and KO-t-Bu (28.7 g) in DMSO (250 ml) as shown above. The residue was dissolved in acetone (10 ml) and loaded onto a silica column (600 g silica gel, packed with hexanes). The column was eluted first with hexanes (800 ml), followed by 5-20% of acetone in hexanes. The fractions containing product were combined and concentrated to give orange solid 5a (33 g).

### Step 5B: (3 S,11bR)-9,10-Dimethoxy-3-(2-methylpropyl)-1H,2H,3H,4H,6H,7H,11bH-pyrido[2,1-a]isoquinoline-2-carboxylic acid

A 1 gallon pressure reactor was charged with a suspension of 5a (94 g, 286 mmol) in methanol (940 ml) and NaOH (343 g, 8.6 mol) in water (940 ml). This mixture was stirred at 120° C. (internal temp) for 67 h. The mixture was cooled to room temperature and transferred to a round bottom flask. The mixture was concentrated in a rotavap to ˜1 L. The mixture was then adjusted pH to 7 using aqueous 6N HCl under cooling. The mixture was extracted with DCM (2×3 L and 1×2 L). The combined organics were dried over Na₂SO₄ and concentrated to give a dark residue (88 g). The dark residue was taken in acetonitrile (500 ml) and stirred for 30 min. The mixture was filtered and the solid was washed with acetonitrile (50 ml). The solid was dried under vacuum for 2 hours to afford light brown solid (42 g, 49%). This solid was combined with the filtrate and concentrated to a residue. The residue was dissolved in DCM (150 ml) and loaded onto a silica column packed with DCM. The column was eluted with 0-25% of methanol in DCM. The fractions containing product were combined and concentrated to give (3S,11bR)-9,10-Dimethoxy-3-(2-methylpropyl)-1H,2H,3H,4H,6H,7H,11bH-pyrido[2,1-a]isoquinoline-2-carboxylic acid 5b as a pale brown solid (71 g, 71% yield, 92% purity, m/z 348.2 [MH⁺]).

### Step 5C: [(2R,3S,11bR)-9,10-Dimethoxy-3-(2-methylpropyl)-1H,2H,3H,4H,6H,7H,11bH-pyrido[2,1-a]isoquinolin-2-yl]methanol

A 3 L round bottom flask was charged with 5b (73.5 g, 211.5 mmol) and THF (1.48 L). This mixture was stirred and cooled to 10° C. (internal temp). To this mixture was added 1 M LAH in THF (423 ml, 423 mmol) slowly over 20 min keeping the temp below 20° C. The cooling bath was removed, and the mixture was warmed up to room temp. The mixture was heated to 55° C. and stirred for 30 min. The mixture was cooled to room temp and then to 10° C. EtOAc (30 ml) was added slowly to quench un-reacted LAH followed by ethanol (30 ml). Then water (150 ml) was added to this mixture. The mixture was then concentrated to remove most of organic solvents. Then the mixture was diluted with water (700 ml) and DCM (1 L). The suspension was filtered through a pad of celite. The filtered cake was washed with DCM (2×500 ml). The combined filtrates were taken in separatory funnel and the layers separated. The aqueous layer was extracted with DCM (1 L). The combined organics were dried over Na₂SO₄ and concentrated to give a dark residue. The residue was chromatographed on silica column using 0-10% of methanol in DCM as eluent. The fractions containing product were combined and concentrated to afford foamy orange residue. To this residue hexanes (100 ml) was added and concentrated under reduced pressure at 45° C. for 2 h to afford [(2R,3S,11bR)-9,10-Dimethoxy-3-(2-methylpropyl)-1H,2H,3H,4H,6H,7H,11bH-pyrido[2,1-a]isoquinolin-2-yl]methanol (5-1) (also called Compound 5-1 herein) as a pale brown solid (51 g, 72%, 95% HPLC purity by 220 nm, m/z 334.2 [MH⁺]). This material may be further purified by silica gel chromatography using 0-10% of methanol in DCM or ethyl acetate as eluent.

### Step 5D: [(2R,3S,11bR)-9,10-Dimethoxy-3-(2-methylpropyl)-1H,2H,3H,4H,6H,7H,11bH-pyrido[2,1-a]isoquinolin-2-yl]methanol HCl salt

A 2 L round bottom flask was charged with 5-1 (43 g, 129 mmol) and diethyl ether (860 mL). This mixture was stirred and cooled to 15° C. (internal temp). To this mixture was added 2 M HCl in diethyl ether (97 ml, 193 mmol) slowly over 15 min. A white precipitate formed. The cooling bath was removed and the mixture was warmed to room temp. The mixture was then stirred for 45 min. The mixture was filtered and the filtered solid was washed with diethyl ether (100 ml), with MTBE (100 ml) and then with hexanes (100 ml). The solid was then dried in vacuum oven at 40° C. for 18 h. [(2R,3 S,11bR)-9,10-Dimethoxy-3-(2-methylpropyl)-1H,2H,3H,4H,6H,7H,11bH-pyrido[2,1-a]isoquinolin-2-yl]methanol HCl salt (5-1 HCl) was isolated as an off-white solid (44.7 g, 94% yield, m/z 334.2 [MH⁺]).

### Example 2

### Methods for Determining VMAT2 Inhibitory Activity of a Compound

Examples of techniques for determining the capability of a compound to inhibit VMAT2 are provided below.

The procedure is adapted from that described previously (see, e.g., Near, (1986), Mol. Pharmacol. 30: 252-57; Teng, et al., J. Neurochem. 71, 258-65, 1998). Homogenates from human platelets or Sprague-Dawley rat forebrain were prepared by homogenization and then washed by centrifugation as described previously (see, e.g., Hoare et al., (2003) Peptides 24:1881-97). In a total volume of 0.2 mL in low-binding 96-well plates (Corning #3605), twelve concentrations of Compound 5-1 and R,R,R-DHTBZ were competed against 6 nM ³H-dihydrotetrabenazine (American Radiolabeled Chemicals, Kd 2.6 nM) on rat forebrain homogenate (100 μg membrane protein per well) or human platelet homogenate (50 μg membrane protein per well) in VMAT2 binding buffer (Dulbecco's phosphate buffered saline, 1 mM EDTA, pH 7.4). Following incubation at 25° C. for two hours, bound radioligand was collected by rapid filtration onto GF/B glass fiber filters using a Unifilter-96 Harvester (PerkinElmer). Filter plates were pre-treated for 10 minutes with 0.1% polyethyleniminc, and following harvesting the filter plates were washed with 800 μl VMAT2 binding buffer. Bound radioligand was quantified by scintillation counting using a Topcount NXT (PerkinElmer). The results of the competition binding studies are presented below in Table 1 and Table 2.

TABLE 1
Rat Forebrain VMAT2 Affinity from Competition Binding Studies

| Compound | pKi (n) | Ki (nM) |
|---|---|---|
| Compound 5-1 | 8.6 ± 0.1 (2) | 2.6 |
| R,R,R-DHTBZ | 8.7 ± 0.2 (6) | 1.9 |

TABLE 2
Human Platelet VMAT2 Affinity from
Competition Binding Studies

| Compound | pKi (n) | Ki (nM) |
|---|---|---|
| Compound 5-1 | 8.3 ± 0.1 (2) | 5.2 |
| R,R,R-DHTBZ | 8.6 ± 0.3 (3) | 2.6 |

Another technique that may be routinely performed to determine the capability of a compound to inhibit VMAT2 is provided below. The following procedure is adapted from a previously described method (see Teng, et al., J. Neurochem. 71, 258-65, 1998).

Preparation of rat striatal vesicles: Rat striata from three rats are pooled and homogenized in 0.32 M sucrose. The homogenate is then centrifuged at 2,000×g for 10 min at 4° C. and the resulting supernatant is centrifuged at 10,000×g for 30 min at 4° C. The resulting pellet containing the enriched synaptosomal fraction (2 mL) is subjected to osmotic shock by addition of 7 mL of distilled H₂O, and subsequently the suspension is homogenized. The osmolarity is restored by the addition of 0.9 mL of 0.25 M HEPES and 0.9 mL of 1.0 M neutral L-(+)-tartaric acid dipotassium salt buffer (pH 7.5), followed by a 20 min centrifugation (20,000×g at 4° C.). The supernatant is then centrifuged for 60 min (55,000×g at 4° C.) and the resulting supernatant is centrifuged for 45 min (100,000 xg at 4° C.). The resulting pellet is resuspended in 25 mM HEPES, 100 mM L-(+)-tartaric acid dipotassium salt, 5 mM MgCl₂, 10 mM NaCl, 0.05 mM EGTA, pH 7.5 to a protein concentration of 1-2 mg/mL and stored at −80° C. for up to 3 weeks without appreciable loss of binding activity. Immediately before use, the final pellet is resuspended in binding buffer (25 mM HEPES, 100 mM L-(+)-tartaric acid dipotassium salt, 5 mM MgCl₂, 10 mM NaCl, 0.05 mM EGTA, 0.1 mM EDTA, 1.7 mM ascorbic acid, pH 7.4).

[³H]-dihydrotetrabenazine (DHTBZ) Binding: Aliquots of the vesicle suspension (0.16 mL, 15 μg of protein/mL) are incubated with competitor compounds (ranging from 10⁻⁶ to 10⁻¹² M) and 2 nM [³H]-dihydrotetrabenazine (HTBZ; specific activity: 20 Ci/mmol, American Radiolabeled Chemicals, Inc.) for 1 h at room temperature in a total volume of 0.5 mL. The reaction is terminated by rapid filtration of the samples onto Whatman GF/F filters using a Brandel cell harvester. Nonspecific binding is determined using 20 μM tetrabenazine (TBZ). Filters are previously soaked for 2 h with ice-cold polyethyleneimine (0.5%). After the filters are washed three times with the ice-cold buffer, they are placed into scintillation vials with 10 mL scintillation cocktail. Bound radioactivity is determined by scintillation spectrometry.

All U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. Although specific embodiments have been described herein for purposes of illustration, various modifications may be made without departing from the spirit and scope of the invention. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for treating mania in a mood disorder in a subject comprising administering to the subject a selective VMAT2 inhibitor.

2. A method for treating mania in a mood disorder in a subject comprising administering to the subject a VMAT2 inhibitor selected from:
tetrabenazine (3-isobutyl-9,10-dimethoxy-1,3,4,6,7,11b-hexahydro-2H-pyrido[2,1-a]isoquinolin-2-one);
(2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof;
(S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
[(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof; and
3-isobutyl-9,10-d₆-dimethoxy-1,3,4,6,7,11b-hexahydro-2H-pyrido[2,1-a]isoquinolin-2-one (d₆-TBZ); or
an isotopic variant, a pharmaceutically acceptable salt, or a polymorph thereof.

3. The method of claim 1, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant, a pharmaceutically acceptable salt, or a polymorph thereof.

4. The method of claim 3, wherein the VMAT2 inhibitor is (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), or an isotopic variant thereof, or polymorph thereof.

5. The method of claim 1, wherein the mood disorder is bipolar disorder.

6. The method of claim 5, wherein the mania in the mood disorder is hypomania or severe mania.

7. The method of claim 1, wherein the VMAT2 inhibitor is administered at a daily dose of about 5 mg to about 100 mg.

8. The method of claim 1, wherein the VMAT2 inhibitor is administered orally.

9. The method of claim 8, wherein the VMAT2 inhibitor is administered as a tablet or capsule.

10. A pharmaceutical composition for use in treating mania in a mood disorder, said composition comprising a pharmaceutically acceptable excipient and a selective VMAT2 inhibitor.

11. A pharmaceutical composition for use in treating mania in a mood disorder, said composition comprising a pharmaceutically acceptable excipient and a VMAT2 inhibitor selected from:
tetrabenazine (3-isobutyl-9,10-dimethoxy-1,3,4,6,7,11b-hexahydro-2H-pyrido[2,1-a]isoquinolin-2-one);
(2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof;
(S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
[(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof; and
3-isobutyl-9,10-d₆-dimethoxy-1,3,4,6,7,11b-hexahydro-2H-pyrido[2,1-a]isoquinolin-2-one (d₆-TBZ); or
an isotopic variant, a pharmaceutically acceptable salt, or a polymorph thereof.

12. The pharmaceutical composition of claim 1, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant, a pharmaceutically acceptable salt, or a polymorph thereof.

13. The pharmaceutical composition of claim 12, wherein the VMAT2 inhibitor is (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), or an isotopic variant thereof, or polymorph thereof.

14. The pharmaceutical composition of claim 11, wherein the mood disorder is bipolar disorder.

15. The pharmaceutical composition of claim 14, wherein the mania in the mood disorder is hypomania or severe mania.

16. The pharmaceutical composition of claim 11, wherein the pharmaceutical composition is formulated as a dosage form having about 5 mg to about 100 mg of the VMAT2 inhibitor.

17. The pharmaceutical composition of claim 11, wherein the pharmaceutical composition is formulated for oral administration.

18. The pharmaceutical composition of claim 17, wherein the pharmaceutical composition is formulated as a tablet or capsule.

19. A method for treating treatment-refractory obsessive-compulsive disorder (OCD) in a subject comprising administering to the subject a selective VMAT2 inhibitor.

20. A method for treating treatment-refractory OCD in a subject comprising administering to the subject a VMAT2 inhibitor selected from:
tetrabenazine (3-isobutyl-9,10-dimethoxy-1,3,4,6,7,11b-hexahydro-2H-pyrido[2,1-a]isoquinolin-2-one);
(2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof;
(S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
[(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof; and
3-isobutyl-9,10-d₆-dimethoxy-1,3,4,6,7,11b-hexahydro-2H-pyrido[2,1-a]isoquinolin-2-one (d₆-TBZ); or
an isotopic variant, a pharmaceutically acceptable salt, or a polymorph thereof.

21. The method of claim 19, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant, a pharmaceutically acceptable salt, or a polymorph thereof.

22. The method of claim 21, wherein the VMAT2 inhibitor is (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), or an isotopic variant thereof, or polymorph thereof.

23. The method of claim 19, wherein the VMAT2 inhibitor is administered at a daily dose of about 5 mg to about 100 mg.

24. The method of claim 19, wherein the VMAT2 inhibitor is administered orally.

25. The method of claim 24, wherein the VMAT2 inhibitor is administered as a tablet or capsule.

26. The method of claim 19, wherein the frequency or severity of cleaning, hoarding, a counting ritual, a checking ritual, a line-crossing, a prayer ritual, a hand washing ritual, following a strict routine, orderliness, requesting reassurance, or a combination thereof is reduced.

27. A pharmaceutical composition for use in treating treatment-refractory OCD, said composition comprising a pharmaceutically acceptable excipient and a selective VMAT2 inhibitor.

28. A pharmaceutical composition for use in treating treatment-refractory OCD, said composition comprising a pharmaceutically acceptable excipient and a VMAT2 inhibitor selected from:
tetrabenazine (3-isobutyl-9,10-dimethoxy-1,3,4,6,7,11b-hexahydro-2H-pyrido[2,1-a]isoquinolin-2-one);
(2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof;
(S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
[(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof; and
3-isobutyl-9,10-d₆-dimethoxy-1,3,4,6,7,11b-hexahydro-2H-pyrido[2,1-a]isoquinolin-2-one (d₆-TBZ);
or an isotopic variant thereof, a pharmaceutically acceptable salt, or polymorph thereof.

29. The pharmaceutical composition of claim 27, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant, a pharmaceutically acceptable salt, or a polymorph thereof.

30. The pharmaceutical composition of claim 29, wherein the VMAT2 inhibitor is (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), or an isotopic variant thereof, or polymorph thereof.

31. The pharmaceutical composition claim 27, wherein the pharmaceutical composition is formulated as a dosage form having about 10 mg to about 80 mg of the VMAT2 inhibitor.

32. The pharmaceutical composition of claim 27, wherein the pharmaceutical composition is formulated for oral administration.

33. The pharmaceutical composition of claim 32, wherein the pharmaceutical composition is formulated as a solution, tablet or capsule.

34. The pharmaceutical composition of claim 27, wherein the frequency or severity of cleaning, hoarding, a counting ritual, a checking ritual, a line-crossing, a prayer ritual, a hand washing ritual, following a strict routine, orderliness, requesting reassurance, or a combination thereof is reduced.

35. A method for treating a neurological dysfunction associated with Lesch-Nyhan syndrome in a subject comprising administering to the subject a selective VMAT2 inhibitor.

36.-42. (canceled)

43. A pharmaceutical composition for use in treating a neurological dysfunction associated with Lesch-Nyhan syndrome, said composition comprising a pharmaceutically acceptable excipient and a selective VMAT2 inhibitor.

44.-50. (canceled)

51. A method for treating agitation in a subject who has Alzheimer's disease comprising administering to the subject a VMAT2 inhibitor.

52.-59. (canceled)

60. A pharmaceutical composition for use in treating agitation in Alzheimer's disease, said composition comprising a pharmaceutically acceptable excipient and a VMAT2 inhibitor.

61.-68. (canceled)

69. A method for treating Fragile X syndrome or Fragile X-associated tremor-ataxia syndrome in a subject comprising administering to the subject a selective VMAT2 inhibitor.

70.-76. (canceled)

77. A pharmaceutical composition for use in treating Fragile X syndrome or Fragile X-associated tremor-ataxia syndrome, said composition comprising a pharmaceutically acceptable excipient and a selective VMAT2 inhibitor.

78.-84. (canceled)

85. A method for treating autism spectrum disorder (ASD) in a subject comprising administering to the subject a VMAT2 inhibitor.

86.-93. (canceled)

94. A pharmaceutical composition for use in treating autism spectrum disorder (ASD), said composition comprising a VMAT2 inhibitor and a pharmaceutically acceptable excipient.

95.-102. (canceled)

103. A method for treating depression in a mood disorder in a subject comprising administering to the subject a selective VMAT2 inhibitor.

104. A method for treating depression in a mood disorder in a subject comprising administering to the subject a VMAT2 inhibitor selected from:
tetrabenazine (3-isobutyl-9,10-dimethoxy-1,3,4,6,7,11b-hexahydro-2H-pyrido[2,1-a]isoquinolin-2-one);
(2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof;
(S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
[(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof; and
3-isobutyl-9,10-d₆-dimethoxy-1,3,4,6,7,11b-hexahydro-2H-pyrido[2,1-a]isoquinolin-2-one (d₆-TBZ); or
an isotopic variant, a pharmaceutically acceptable salt, or a polymorph thereof.

105. The method of claim 103, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant, a pharmaceutically acceptable salt, or a polymorph thereof.

106. The method of claim 105, wherein the VMAT2 inhibitor is (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), or an isotopic variant thereof, or polymorph thereof.

107. The method of claim 103, wherein the mood disorder is bipolar disorder.

108. The method of claim 103, wherein the mood disorder is major depressive disorder.

109. The method of claim 103, wherein the VMAT2 inhibitor is administered at a daily dose of about 5 mg to about 100 mg.

110. The method of claim 103, wherein the VMAT2 inhibitor is administered orally.

111. The method of claim 110, wherein the VMAT2 inhibitor is administered as a tablet or capsule.

112. A pharmaceutical composition for use in treating depression in a mood disorder, said composition comprising a pharmaceutically acceptable excipient and a selective VMAT2 inhibitor.

113. A pharmaceutical composition for use in treating depression in a mood disorder, said composition comprising a pharmaceutically acceptable excipient and a VMAT2 inhibitor selected from:
tetrabenazine (3-isobutyl-9,10-dimethoxy-1,3,4,6,7,11b-hexahydro-2H-pyrido[2,1-a]isoquinolin-2-one);
(2R,3R,11bR)-3-isobutyl-9,10-dimethoxy-1,3,4,6,7,11b-hexahydro-2H-pyrido[2,1-a]isoquinolin-2-ol (R,R,R DHTBZ), or a precursor thereof;
(S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester;
[(2R,3S,11bR)-9,10-Dimethoxy-3-(2-methylpropyl)-1H,2H,3H,4H,6H,7H,11bH-pyrido[2,1-a]isoquinolin-2-yl]methanol, or a precursor thereof; and
3-isobutyl-9,10-d₆-dimethoxy-1,3,4,6,7,11b-hexahydro-2H-pyrido[2,1-a]isoquinolin-2-one (d₆-TBZ); or
an isotopic variant, a pharmaceutically acceptable salt, or a polymorph thereof.

114. The pharmaceutical composition of claim 112, wherein the VMAT2 inhibitor is (S)-2-Amino-3-methyl-butyric acid (2R,3R,11bR)-3-isobutyl-9,10-dimethoxy-1,3,4,6,7,11b-hexahydro-2H-pyrido[2,1-a]isoquinolin-2-yl ester or an isotopic variant, a pharmaceutically acceptable salt, or a polymorph thereof.

115. The pharmaceutical composition of claim 114, wherein the VMAT2 inhibitor is (S)-(2R,3R,11bR)-3-isobutyl-9,10-dimethoxy-2,3,4,6,7,11b-hexahydro-1H-pyrido[2,1-a]isoquinolin-2-yl 2-amino-3-methylbutanoate di(4-methylbenzenesulfonate), or an isotopic variant thereof, or polymorph thereof.

116. The pharmaceutical composition of claim 113, wherein the mood disorder is bipolar disorder.

117. The pharmaceutical composition of claim 116, wherein the mood disorder is major depressive disorder.

118. The pharmaceutical composition of claim 112, wherein the pharmaceutical composition is formulated as a dosage form having about 5 mg to about 100 mg of the VMAT2 inhibitor.

119. The pharmaceutical composition of claim 112, wherein the pharmaceutical composition is formulated for oral administration.

120. The pharmaceutical composition of claim 119, wherein the pharmaceutical composition is formulated as a tablet or capsule.

121. A method for treating chorea-acanthocytosis in a subject comprising administering to the subject a selective VMAT2 inhibitor.

122.-128. (canceled)

129. A pharmaceutical composition for use in treating chorea-acanthocytosis, said composition comprising a pharmaceutically acceptable excipient and a selective VMAT2 inhibitor.

130.-136. (canceled)

137. A method for treating Rett syndrome in a subject comprising administering to the subject a selective VMAT2 inhibitor.

138.-144. (canceled)

145. A pharmaceutical composition for use in treating Rett syndrome, said composition comprising a pharmaceutically acceptable excipient and a selective VMAT2 inhibitor.

146.-152. (canceled)
